(19) United States
(12) Patent Application Publication
(10) Pub. No.: US 2014/0030745 A1
(43) Pub. Date: Jan. 30, 2014

(54) ASSAY KIT AND ANALYSIS METHOD

(71) Applicant: NATIONAL CENTRAL UNIVERSITY, Jhongli City (TW)

(72) Inventors: Chien-Sheng CHEN, Jhongli City (TW); Tien-Yu HO, Zhongli City (TW)

(73) Assignee: NATIONAL CENTRAL UNIVERSITY, Jhongli City (TW)

(21) Appl. No.: 13/839,392

(22) Filed: Mar. 15, 2013

(30) Foreign Application Priority Data
Jul. 24, 2012 (TW) 101126604

(51) Int. Cl. G01N 33/543

(52) U.S. Cl. CPC G01N 33/54313
USPC 435/7.94; 436/501; 435/7.1; 435/7.92

(57) ABSTRACT

An assay kit for reacting with an analyte includes a plurality of reaction vessels and a plurality of micro beads. Each of the reaction vessels contains a filter membrane with a plurality of pores, and the diameter of each micro bead is greater than that of each pore. Each of the micro beads is directly or indirectly bound to the analyte, the competitor of the analyte or the identifying molecule. Additionally, an analysis method applied with the assay kit is also disclosed.

### CROSS REFERENCE TO RELATED APPLICATIONS

This Non-provisional application claims priority under 35 U.S.C. §119(a) on Patent Application No(s). 101126604 filed in Taiwan, Republic of China on Jul. 24, 2012, the entire contents of which are hereby incorporated by reference.

### BACKGROUND OF THE INVENTION

1. Field of Invention

The present invention relates to an assay kit and an analysis method.

2. Related Art

Bio detection is one of the major methods for environmental monitoring, scientific analyzing, medical diagnosis, industrial quality control, and food safety. The analysis methods utilizing the specific identification of the identifying molecule with respect to the analyte has been widely applied to many techniques for a long time. The common analysis methods include, for example, microtiter plate based assay, lateral flow immunochromatographic assay, and bead based assay.

The bead based assay is benefit in uniform distribution of solid phase and liquid phase molecules in their mixture. In the bead based assay, a flow cytometer and different fluorescent marked beads are used to detect targets. Otherwise, the bead based assay may also utilize magnetic beads for purification. In detailed, the magnetic beads are combined with the substances to be analyzed in specific, and then the magnetic beads are separated for the following analyzing and detecting.

However, the bead based assay needs to operate the flow cytometer for analyzing or mark the beads with different analyzing functions, so that the analyzing process is complex and high costly. Thus, the conventional bead based assay can not fit the demands of large amount and rapid analysis. Regarding to the magnetic beads, they can be separated and purified by magnetic force, so that using the magnetic beads can significantly improve the convenience, sensitivity, specificity, speed, and simplification of the bead based assay. Unfortunately, since it is necessary to apply magnetic force to separate the magnetic beads from other substances. Therefore, the operations are complex and some magnetic beads may be lost when the applied magnetic force is not uniform or is insufficient.

Thus, it is an important subject to provide an assay kit and an analysis method that have simple procedures and low cost and can rapidly analyze large amount of samples.

### SUMMARY OF THE INVENTION

In view of the foregoing subject, an objective of the present invention is to provide an assay kit and an analysis method that have simple procedures and low cost and can rapidly analyze large amount of samples.

To achieve the above objective, the present invention discloses an assay kit, which is configured for reacting with an analyte and comprises a plurality of reaction vessels and a plurality of micro beads. Each reaction vessel contains a filter membrane with a plurality of pores. The diameter of each micro bead is greater than that of each pore, and each micro head is directly or indirectly bound to the analyte, competitor thereof or the identifying molecule.

In one embodiment, the diameter of each of the pores is between 1 nm and 1 cm.

In one embodiment, the beads are made of glass, latex, rubber, magnet, resin, metal, ceramic, polysaccharide, plastic, or silicon.

In one embodiment, the analyte, the competitor, or the identifying molecules thereof is protein, peptide, nucleic acid, saccharide, compound, cell, or microorganism.

In one embodiment, the identifying molecule is configured for binding with the analyte or the competitor thereof.

In one embodiment, the assay kit further comprises a first ligand for directly or indirectly binding the analyte, the competitor thereof, or an identifying molecule on the beads.

In one embodiment, the assay kit further comprises a second ligand for connecting with the analyte, the competitor thereof, or the identifying molecule. The second ligand is bound with the first ligand by antigen-antibody binding, protein-cofactor binding, nucleic acid-nucleic acid binding, nucleic acid-saccharide binding, nucleic acid-compound binding, protein-protein inhibitor binding, protein-saccharide binding, protein-lipid binding, protein-compound binding, enzyme-enzyme receiver binding, or protein-nucleic acid binding.

In one embodiment, the assay kit further comprises a plurality of signal molecules for binding with the analyte, the competitor thereof, or the identifying molecule.

In one embodiment, the signal molecule comprises enzyme, enzyme substrate, chromogenic agent, radioactive substance, nano-lipid particle, or metallic compound.

In one embodiment, the reaction vessel comprises at least a filter plate or at least a column.

In one embodiment, the assay kit further comprises a scaler disposed at a downstream of the filter membrane.

To achieve the above objective, the present invention also discloses an analysis method applied to an assay kit for reacting with an analyte. The assay kit comprises a plurality of reaction vessels and a plurality of micro beads, and each reaction vessel contains a filter membrane with a plurality of pores. The analysis method comprises the steps of: adding a solution containing the analyte, a competitor of the analyte or the identifying molecules into each of the reaction vessels so that the micro beads directly or indirectly bind with the analyte, the competitor thereof or the identifying molecules; adding a plurality of signal molecules for binding with the analyte, the competitor thereof or the identifying molecules; filtering away the solution in the reaction vessel; and detecting an intensity of a signal generated by the signal molecules.

In one embodiment, the diameter of each of the micro beads is greater than that of each of the pores.

In one embodiment, at least one of the identifying molecule is configured for binding with the analyte or the competitor thereof and directly or indirectly binding with the micro beads.

In one embodiment, the analysis method further comprises a step of disposing at least a first ligand on the surfaces of the micro beads, wherein the first ligand directly or indirectly binds with the analyte, the competitor thereof, or an identifying molecule.

In one embodiment, the analysis method further comprises a step of disposing a second ligand on the analyte, the competitor thereof, or the identifying molecule, wherein the second ligand is bound with the first ligand.

In one embodiment, the second ligand is bound with the first ligand by antigen-antibody binding, protein-cofactor binding, nucleic acid-nucleic acid binding, nucleic acid-saccharide binding, nucleic acid-compound binding, protein-protein inhibitor binding, protein-saccharide binding, protein-lipid binding, protein-compound binding, enzyme-enzyme receiver binding, or protein-nucleic acid binding.

In one embodiment, the analysis method further comprises a step of disposing a sealer at a downstream of the filter membrane.

In one embodiment, the analysis method further comprises a step of removing the sealer to allow the solution to flow through the pores.

As mentioned above, in the assay kit of the invention, the diameter of each micro bead is greater than that of each pore, so that the beads directly or indirectly binding with the analyte, the competitor or the identifying molecules thereof can be rapidly separated by filtering for the following quantitative analysis. Thus, the complex procedures for providing magnetic force to separate the magnetic beads in the conventional art can be omitted. The assay kit of the invention also has the advantages of easily analyzing, lower equipment cost, and applicable to various analysis methods. Besides, the reaction vessels can be designed in different sizes according to the desired analysis, so they can be applied to high throughput analyze. In addition, the present invention also discloses an analysis method applied with the above-mentioned assay kit, which adapts to indirect ELISA, competitive ELISA, and sandwich ELISA. Compared with the conventional analysis method, the analysis method of the invention can more rapidly and easily detect the analyte.

### BRIEF DESCRIPTION OF THE DRAWINGS

The invention will become more fully understood from the detailed description and accompanying drawings, which are given for illustration only, and thus are not limitative of the present invention, and wherein:

FIGS. 1A to 1D are schematic diagrams shown one of the reaction vessels in the assay kit of the invention and its modifications;

FIG. 2A is a sectional view of an assay kit according to an embodiment of the invention;

FIG. 2B is a top view of an assay kit according to another embodiment of the invention;

FIG. 3 is flow chart showing an analysis method of the invention;

FIGS. 4A to 4E are schematic diagrams of an assay kit of the invention, which is applied with the analysis method of the invention; and

FIGS. 5A and 5B are graphs showing the analyzing results of gentamycin by the assay kit and analysis method of the invention.

### DETAILED DESCRIPTION OF THE INVENTION

The present invention will be apparent from the following detailed description, which proceeds with reference to the accompanying drawings, wherein the same references relate to the same elements.

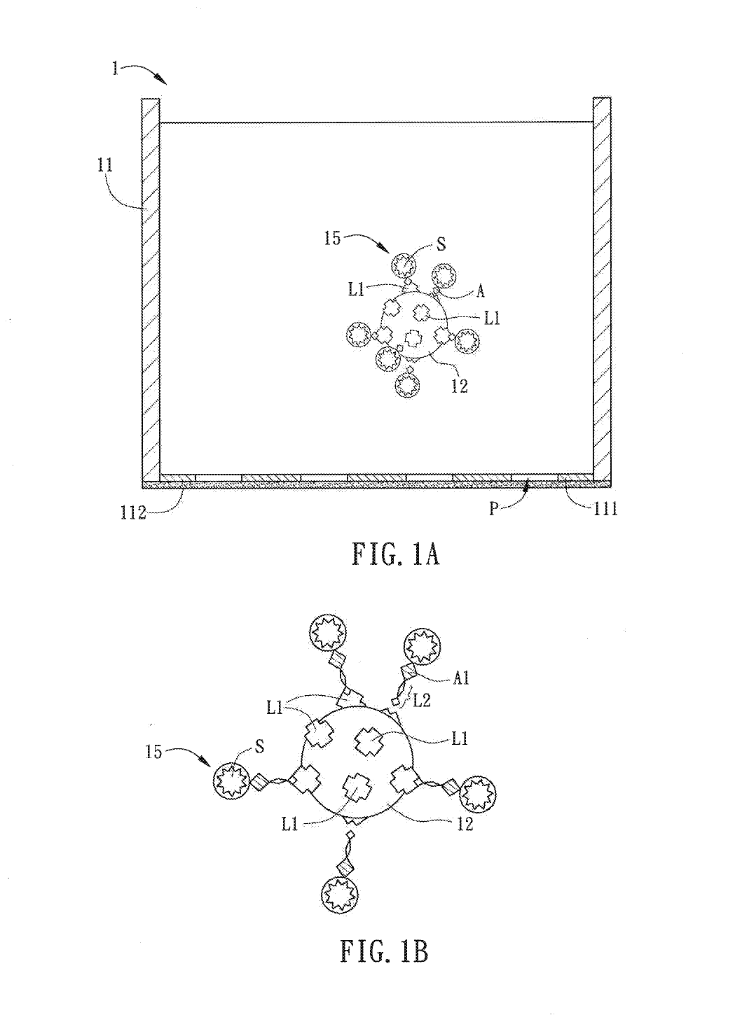

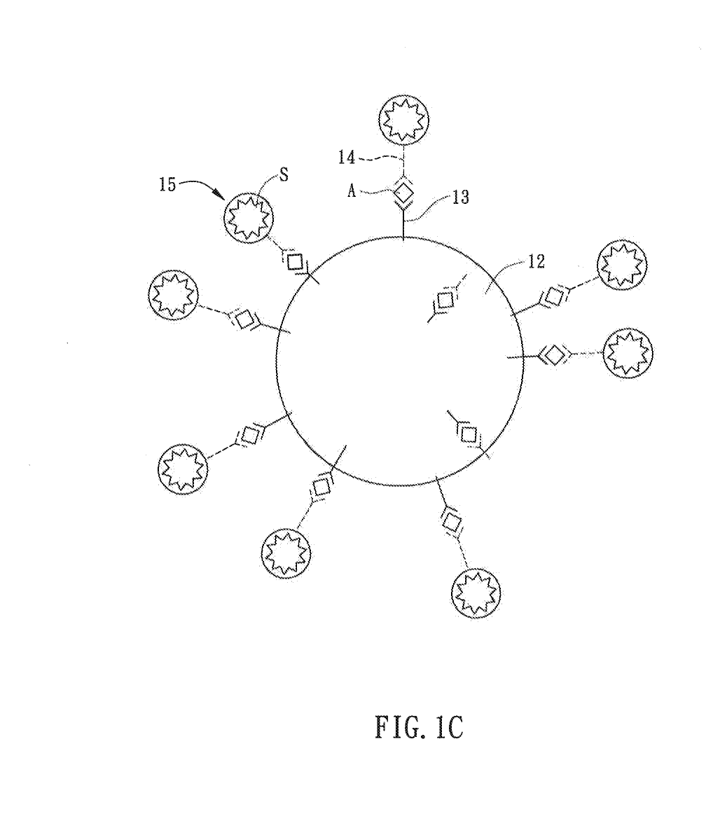

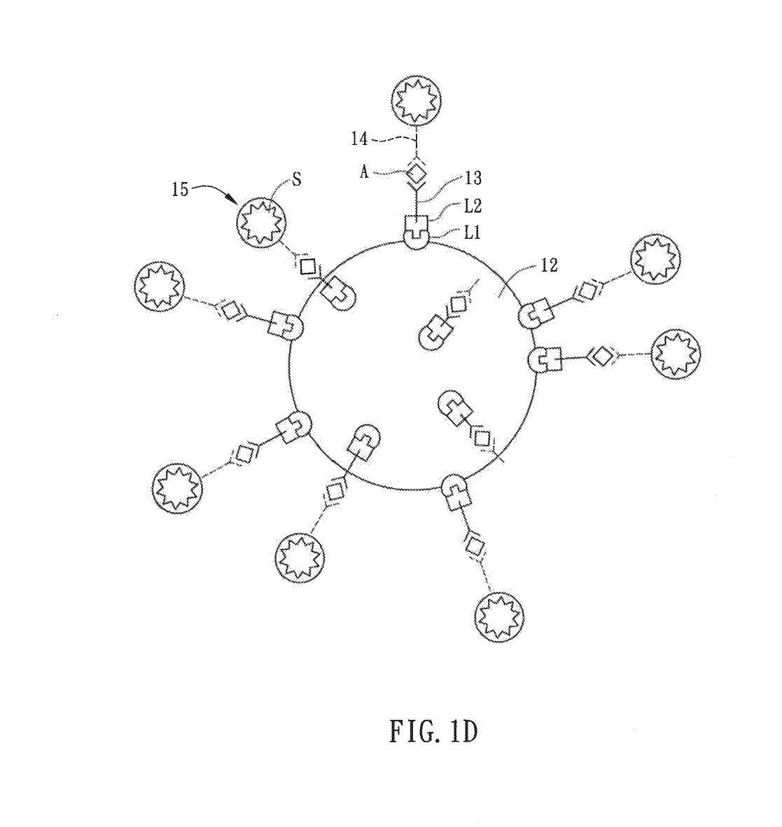

The present invention discloses an assay kit for reacting with an analyte to detect the concentration of the analyte. The assay kit of the invention can be applied to chemical detection and immunization detection. FIG. 1A is a schematic diagram shown a reaction vessel in an assay kit of the invention. Referring to FIG. 1A, the assay kit 1 of the invention includes a plurality of reaction vessels 11 and a plurality of micro beads.

Each reaction vessel 11 contains a filter membrane 111 disposed at the internal space, bottom, or side wall of the reaction vessel 11. In one embodiment, the filter membrane 111 is disposed at the bottom edge of the reaction vessel 11 and is located around the exit of the reaction vessel 11. The filter membrane 111 can be made of cellulose acetate or polyester sulfone. In addition, the filter membrane 111 of the invention has a plurality of pores P, and the diameter of each pore P is between 1 nm and 1 cm, and preferably between 0.1 μm and 10 μm. The distances between the pores P can be the same or different.

The reaction vessel 11 and the filter membrane 111 define a space for carrying out the desired operation and reaction, which contains a plurality of micro beads 12 and/or the competitors of the analyte A. In this case, each reaction vessel 11 is configured with one filter membrane 111. Of course, the reaction vessels 11 of the assay kit 1 may share a single large filter membrane 111, and each reaction vessel 11 is configured corresponding to a small part of the filter membrane 111.

The diameter of each micro bead 12 is greater than that of each pore P of the filter membrane 111, so that the micro beads 12 can be remained inside the reaction vessel 11 after the filter step. Preferably, the diameter of the micro bead 12 is between 2 nm and 2 cm. In this embodiment, the beads 12 can be made of glass, latex, rubber, magnet, resin, metal, ceramic, polysaccharide, plastic, or silicon, and the shape of the micro bead 12 is, for example but not limited to, spherical, oval, square, or any other irregular shape. Herein, the micro bead 12 is a spherical structure for providing a larger surface to bind with the analyte A. Moreover, the spherical surface of the micro beads 12 can easily contact with the sample solution containing the analyte A sufficiently, thereby increasing the reaction uniformity.

The micro beads of the invention can directly or indirectly bind with the analyte, its competitor(s) or the identifying molecules. Herein, the term “directly bind” means that the micro beads bind with the analyte, its competitor or the identifying molecules without relying on other interposed components or molecules On the other hand, the term “indirectly bind” means that the micro beads bind with the analyte, its competitor or the identifying molecules with relying on some interposed components or molecules. The suitable analyte, competitor or identifying molecules of the assay kit of the invention includes protein, peptide, nucleic acid, saccharide, compound, cell, microorganism, or small molecule. For example, the small molecule can be environmental hormones (e.g. melamine, dioxin, ractopamine, phthalate, etc.), physiological metabolites (e.g. glucose, uric acid, etc.), or food additives. The compound can be medical compositions or some small molecules (molecular weight smaller than 1000). The analyte can be prepared by separating from organisms, extraction, preservation, or synthesizing. In other words, the analyte can be a substance to be analyzed within any sample.

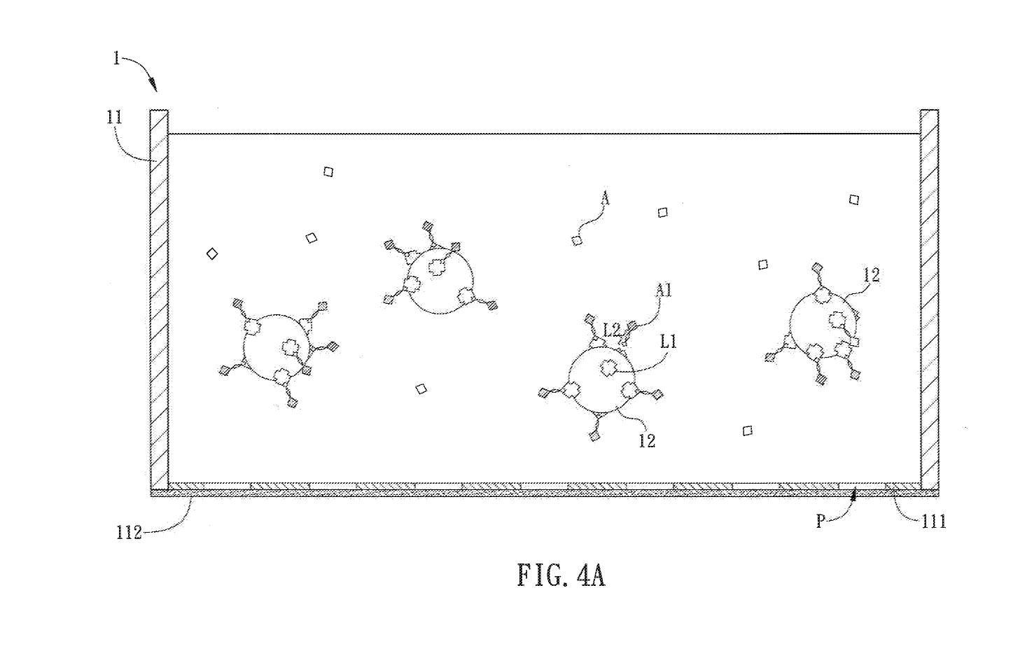

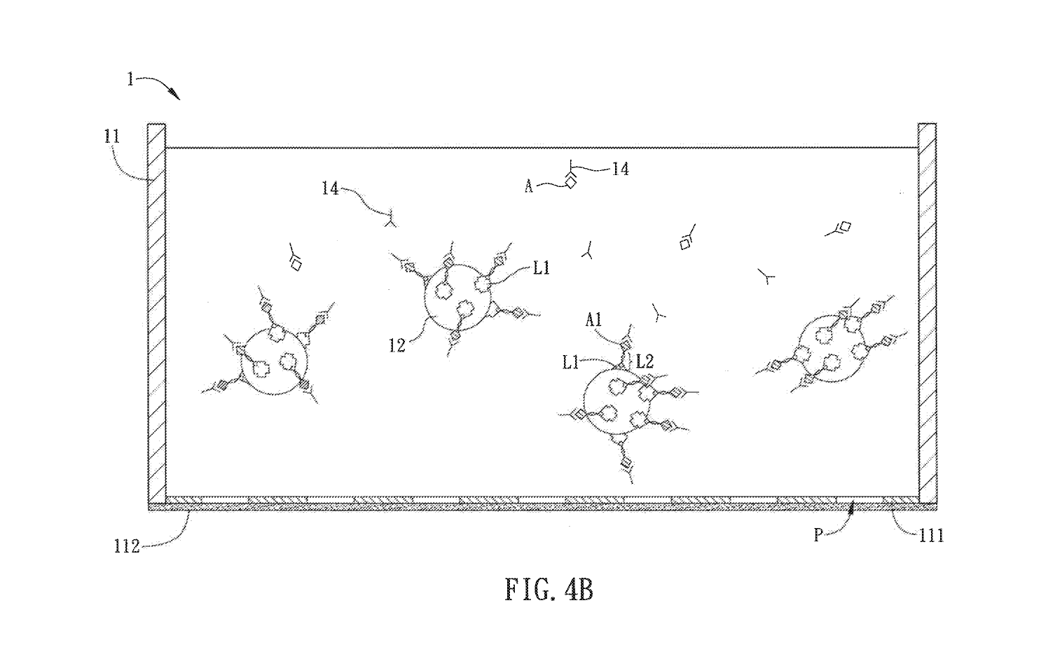

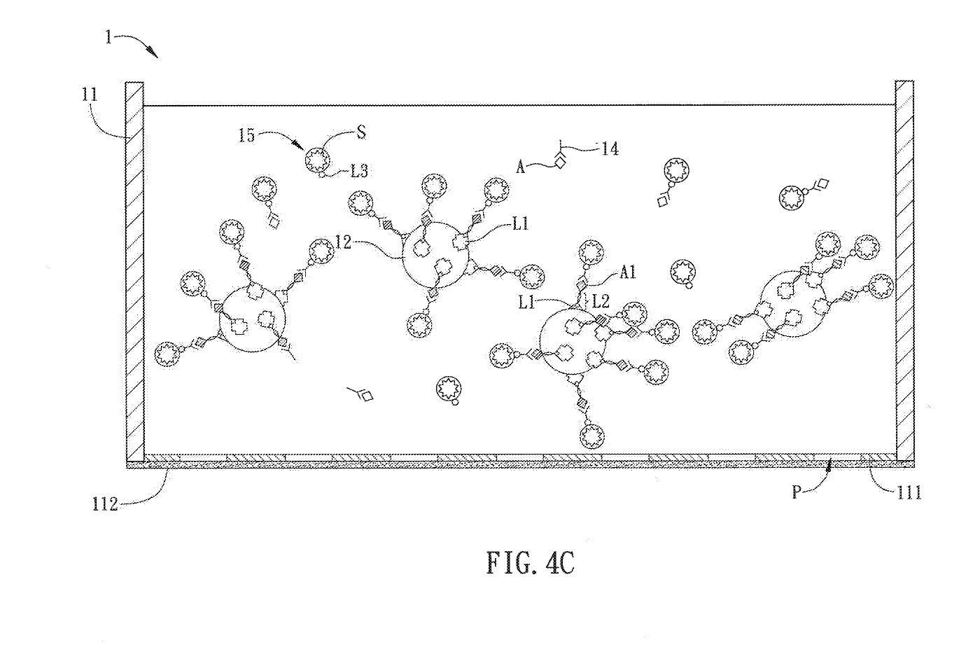

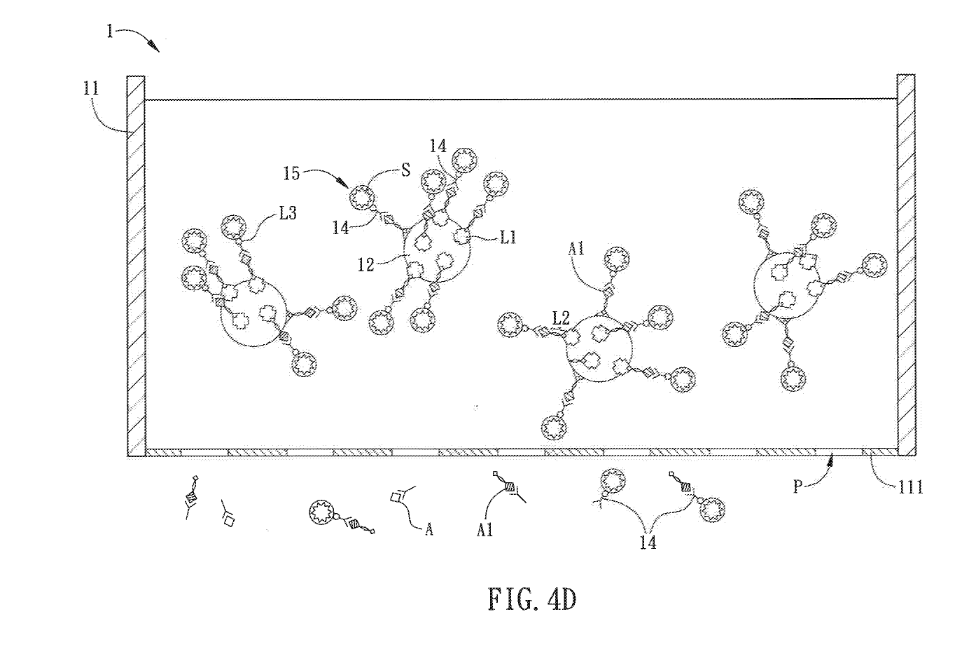

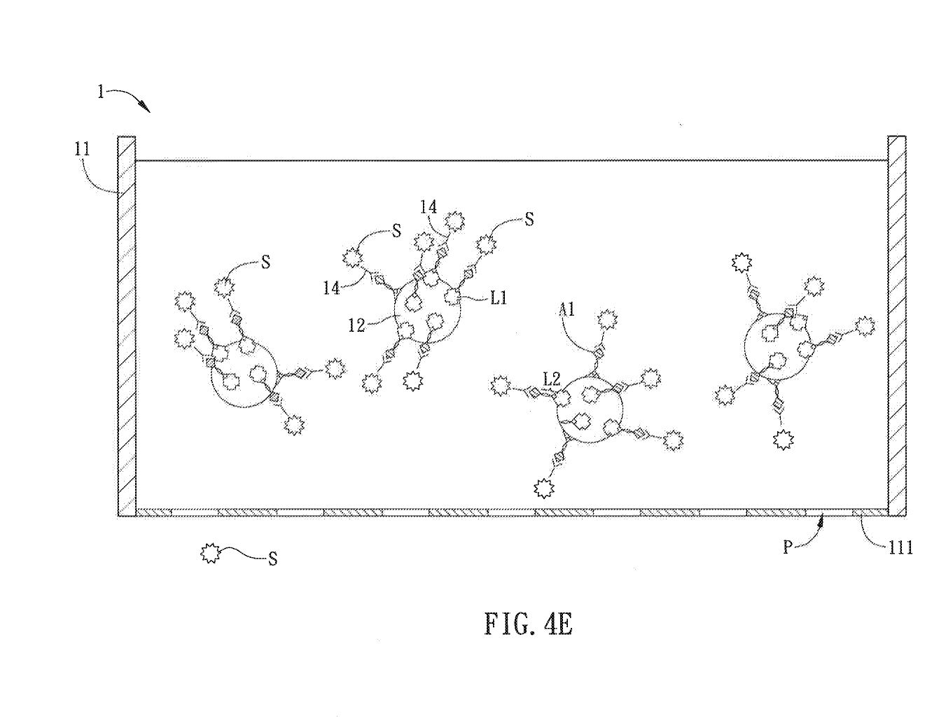

The combination and binding between the elements may be various in different analysis methods. For example, in an indirect ELISA as shown in FIG. 1A, the micro bead 12 is indirectly bound with the analyte A through a first ligand L1. Otherwise, in a competitive ELISA as shown in FIG. 4A, except for the analyte A, which is indirectly bound with the micro bead 12 through a first ligand L1, the competitor A1 of the analyte A contained in the sample and having similar structure as the analyte A can also be indirectly bound with the micro bead 12. The competitor A1 is added for analyzing the concentration of the analyte A. The competitor A1 can be any substance that can bind with an identifying molecule capable of specific identifying the analyte. In other words, the analyte and its competitor can compete with the sites of the identifying molecule (e.g. an antibody) to be bound. The competitor may be a substance, such as a protein, peptide, nucleic acid, saccharide, compound, cell, microorganism, or small molecule, which has the same property or partially similar structure as the analyte. In general, the analyte A or the competitor A1 has a specific complementary structure or charges, so that it can be directly or indirectly bound with the micro bead 12. Otherwise, the analyte A or the competitor A1 may have a specific functional group that can directly or indirectly bind with the micro bead 12.

The analyte, its competitor or the identifying molecules can directly bind with the micro bead by charges attraction, structural engagement, bonding of specific modified functional group. In this embodiment, the micro bead is made of resin carrying negative charges, while the analyte is a fluorescent protein carrying positive charges. When the sample solution containing the analyte is injected into the reaction vessel, the analyte is absorbed by the micro bead and the residual substances can be filtered away. Since the analyte is fluorescent, it is possible to directly detect the concentration of the analyte.

Alternatively, the micro beads can be indirectly bound with the analyte, the competitor thereof and the identifying molecules. For example, in order to increase the binding between the elements, such as the micro bead, analyte, competitor and identifying molecule, or when the above-mentioned structural engagement or functional group can not properly bind the identifying molecule, analyte or competitor with the micro bead, a ligand with better binding affinity or stability is provided in the assay kit. The ligand can be disposed on the surface of at least one of the micro bead, the identifying molecule, analyte, and its competitor, thereby improving the connection between any two elements thereof. For instance, the ligand can be provided to bind two elements; otherwise, two elements can be bound with two different ligands, and the two ligands are further connected. To be noted, each of the elements is not limited to bind with a single ligand, and of course, one element can be bound with two or more ligands (or a ligand set) for further connecting with another element. The configuration of the ligand(s) depends on the analysis purpose such as for different analysis methods (e.g. indirect ELISA, competitive ELISA, or sandwich ELISA).

Referring to FIG. 1A, the assay kit 1 of one embodiment of the invention further includes a first ligand L1, which is configured for directly or indirectly binding with the analyte A or competitor A1, thereby connecting the analyte A or competitor A1 to the micro bead 12. For example, when the bonding force between the micro bead 12 and the analyte A or its competitor is weak, it is preferred to provide a first ligand L1 having better connection with the micro bead 12. The first ligand L1 is connected to the micro bead 12 and further provides a structure for combining with the analyte A, so that the analyte A can be indirectly bound with the micro bead 12 stably through the first ligand L1.

Referring to FIG. 1B, the assay kit of the invention can further include a second ligand L2 for binding with the first ligand L1. The second ligand L2 is configured for connecting with the identifying molecules, the analyte or its competitor A1, so that identifying molecules, the analyte or its competitor A1 can be indirectly bound with the micro bead 12. In practice, the first ligand L1 is directly connected to the micro bead 12, and the second ligand L2 is bound with the competitor A1 and then engaged with the first ligand L1. This mechanism can properly improve the connection between the competitor A1 and the micro bead 12. Of course, in this embodiment of using the first ligand L1 and the second ligand L2 to bind the competitor A1 on the micro bead 12, the second ligand L2 may be engaged with the first ligand L1 and bound with the competitor A1, and the first ligand L1 is then connected with the micro bead 12. Otherwise, the first ligand L1 is connected with the micro bead 12 and then the second ligand L2 is added to bind with the first ligand L1. Afterwards, the competitor A1 is added. Alternatively, the first ligand L1 and the second ligand L2 are connected in advance, and the other end of the first ligand L1 is bound with the micro bead 12. Afterwards, the competitor A1 is added. This invention is not limited to the above.

The first ligand L1 and the second ligand L2 can be bound by antigen-antibody binding (e.g. the binding of gentamycin and anti-gentamycin antibody), protein-cofactor binding (e.g. the binding of biotin and strapavidin or avidin), nucleic acid-nucleic acid binding (e.g. the binding of nucleic acid and nucleic acid aptamers), nucleic acid-saccharide binding, nucleic acid-compound binding, protein-protein inhibitor binding (e.g. the binding of tyrosine phosphatase and gefitinib), protein-saccharide binding, protein-lipid binding, protein-compound binding, enzyme-enzyme receiver binding, or protein-nucleic acid binding.

To be noted, the first ligand L1 may be bound with the analyte A, the competitor A1 or the second ligand L2 depending on the selected substances. In addition, according to the structure or the amount and property of the functional group of the first ligand L1, it may be bound with the second ligand 12 or the analyte A or competitor A1 with at least one second ligand L2. Similarly, one ligand L2 may be bound with one or more analyte A or competitor A1. In this case, a single first ligand L1 can only bind with one analyte A or competitor A1, and a single second ligand L2 can only bind with one analyte A or competitor A1.

In another embodiment, the assay kit is applied to sandwich ELISA. As shown in FIG. 1C, the identifying molecule 13 is a capture antibody for binding with the analyte A, and the identifying molecule 13 is directly connected to the surface of the micro bead 12.

Similarly, the micro bead 12 and the identifying molecule 13 can be bound with the first ligand L1 and the second ligand L2, respectively, and then the first ligand L1 is bound with the second ligand L2 so as to indirectly connect the identifying molecule 13 on the micro bead 12. In this embodiment, the first ligand L1 and the second ligand L2 may be bound by their complementary structures or corresponding functional groups. To be noted, the assay kit of the invention may contain one or more identifying molecules according to the adopted analysis method or the analysis requirement. Referring to FIGS. 1C and 1D, the assay kit may further include another identifying molecule 14, which is a detection antibody. After the identifying molecule 13 binds with an analyte A, the other identifying molecule 14 can identify the analyte A. The identifying molecules 13 and 14 can identify the structures of two different parts of the analyte A, respectively. The operations and theory of the sandwich ELISA are familiar to those skilled persons, so the detailed descriptions thereof will be omitted hereinafter.

Referring to FIGS. 1A to 1D, after the first ligand L1 is directly or indirectly bound with the analyte A, its competitor A1, or the identifying molecule 13, the assay kit 1 of the invention further includes a plurality of signal molecules S for directly or indirectly binding with the analyte A, its competitor A1 or the identifying molecule 13 for quantifying the concentration of the analyte A. In practice, the concentration of the signal molecules S and the concentration of the analyte A are in positive or negative correlation, so that the concentration of the analyte A can be measured by detecting the signal intensity from the activated signal molecules S. The signal molecule S includes enzyme, enzyme substrate, chromogenic agent, radioactive substance, nano-particle, or metallic compound. The enzyme includes luciferase, β-galactosidase, horseradish peroxidase, or alkaline phosphatase, which can release fluorescent signal after adding substrates. The enzyme substrate can connect with the enzyme and then be activated to generate signals. The chromogenic agent is, for example, fluorescein isothiocynate, rhodamine, phycoerythrin, fluorescent protein, sulforhodamine B (SRB), or chemiluminescence (cold light). The radioactive substance includes ¹²⁵I, ³⁵S, or ¹¹¹Tc. The metallic compound is, for example, a Ru complex. When the signal molecule S is a liposome covered with the signal molecules S, the lipid structure is embedded with the signal molecules carrying the desired signals. Some signal molecules S are added or activated right before the quantification analysis, so that the signal molecules S can be effectively detected during their active period so as to prevent the quantification error. The method for detecting the signal molecules S may be various with respect to different signal molecules. For example, the method for detecting the signal molecules S may rely on measuring the absorbance or radiation intensity of the signals as well as the intensity of the signals by proper equipment. However, the operations and theory of the detecting methods are familiar to those skilled persons, so the detailed descriptions thereof will be omitted hereinafter.

In one embodiment of the invention, the signal molecule S is indirectly connected with the analyte A as shown in FIGS. 1C and 1D, wherein the signal molecule S is encapsulated by a liposome so as to form a signal complex 15. The signal complex 15 can direct bind with the analyte A or indirectly bind with the analyte A through an identifying molecule 14. The liposome is removed or broken to expose the signal molecule S before the quantification, and then the concentration of the signal molecules S is measured to determine the concentration of the analyte A.

Similarly, the signal molecule S may further include a ligand (not shown) for improving the binding between the signal molecule S and the analyte A. The ligand can directly or indirectly connect with the signal molecule S or the signal complex 15. The signal molecule S can have better affinity to the analyte A through the ligand so as to firmly bind the identifying molecule with the analyte A. The descriptions and preparations of connecting the signal molecules S and liposome and binding the signal molecule S with the ligand will be illustrated hereinafter. Except for the liposome, the signal molecule S of the invention may also bind with the analyte through other elements, such as an antibody or a linker that can specific bind with the analyte.

With reference to FIGS. 1A to 1D, the assay kit of the invention further includes a sealer 112 disposed at the downstream of the filter membrane 111. The sealer 112 can temporarily seal the pores P of the filter membrane 111, so that the substances (e.g. the micro bead 12 and first ligand L1, the first ligand L1 and analyte A, competitor A1 or second ligand L2, or the signal molecule S and analyte A, competitor A1 or identifying molecule 14) in the reaction vessel can have enough time to perform specific binding. After the reaction is completed, the sealer 112 is removed to filter away the redundant solution. In one embodiment, the sealer 112 is a thin film or a cap, and it can be made of metal, latex or plastic.

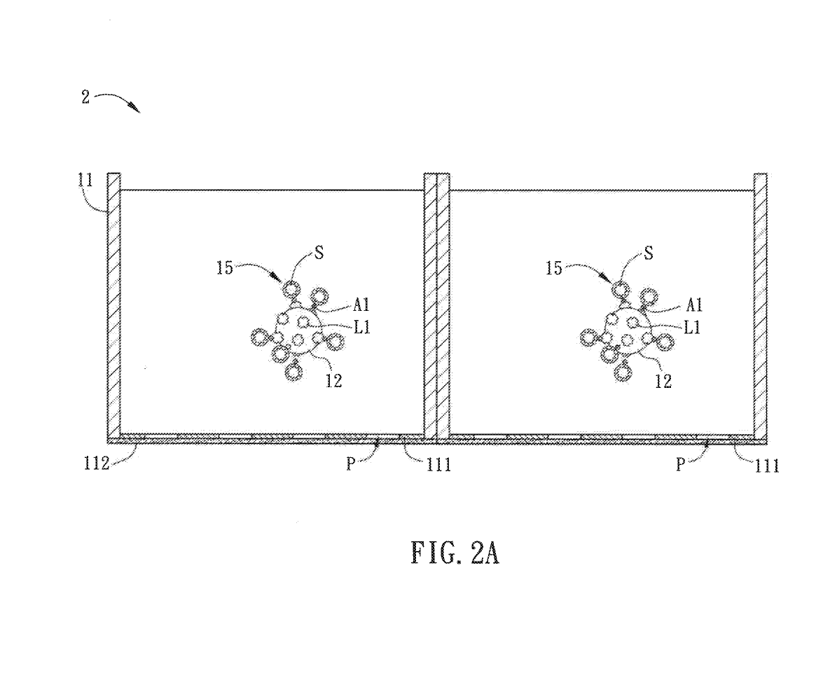

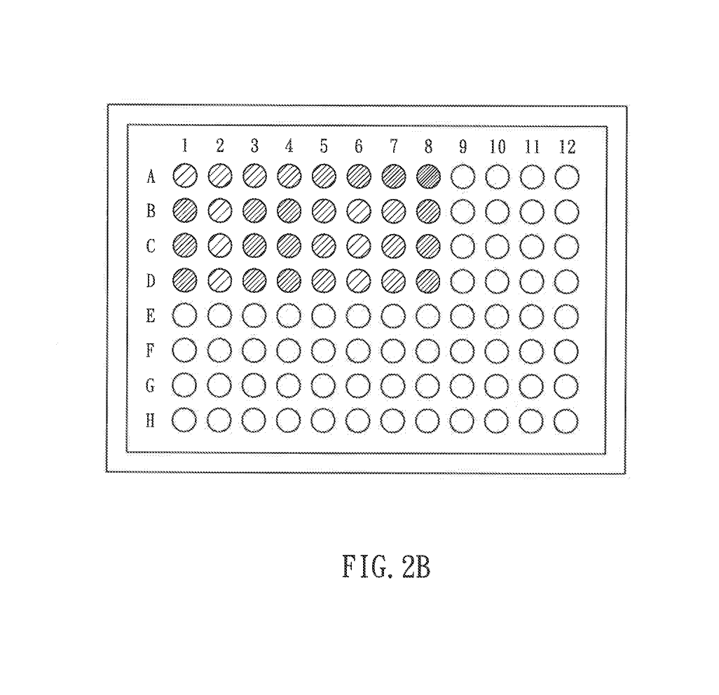

As mentioned above, the reaction vessel 11 has a filter membrane 111, so each reaction vessel of the assay kit of the invention is formed as at least one filter plate or at least one column. When the reaction vessel is a filter plate, it is possible to perform multiple different analysis methods or to analyze different analytes at the same time. The reaction vessels can be assembled side by side on a plane as shown in FIG. 2A so as to form an assay kit 2 containing reaction vessels 11 of different numbers. Preferably, the reaction vessels are arranged in a matrix so as to form, for example, a 96-well filter plate, 384-well filter plate, or 1536-well filter plate. The depth of the reaction chamber (e.g. 1 μl to 100 ml) in each filter plate or column is adjustable according to the size of the sample to be analyzed. Since the assay kit 1 of the invention has a plurality of reaction vessels 11, it can perform various kind detections and analysis, including to establish a standard concentration curve based on a standard sample, or to repeat detections with the same sample.

For example, FIG. 2B is a top view of an assay kit (96-well filter plate). Herein, the wells A1 to A8 are added with the standards of different concentrations, which are measured to establish a standard curve for the following concentration analysis of the analyte. Eight different analytes are added into the wells B1 to B8, respectively, and the wells C1 to C8 and the wells D1 to D8 are repeated operations as the wells B1 to B8. The analyzing results of the wells B1 to B8, C1 to C8, and D1 to D8 are collected to estimate an average concentration with reference to the standard curve. Accordingly, the assay kit of the invention can not only establish the standard curve, but also perform multiple repeated analyses. Besides, since the space of each reaction vessel can be adjusted based on the sample to be analyzed, the assay kit can be applied to high throughput analysis.

The present invention also discloses an analysis method applied to an assay kit for reacting with an analyte. The assay kit includes a plurality of reaction vessels and a plurality of micro beads, and each reaction vessel contains a filter membrane with a plurality of pores. The analysis method includes the steps of: adding a solution containing the analyte, competitor of the analyte or the identifying molecules into each of the reaction vessels so that the micro beads directly or indirectly bind with the analyte, the competitor thereof or the identifying molecules (step S1); adding a plurality of signal molecules for binding with the analyte, the competitor thereof or the identifying molecules (step S2); filtering away the solution in the reaction vessel (step S3); and detecting an intensity of a signal generated by the signal molecules (step S4).

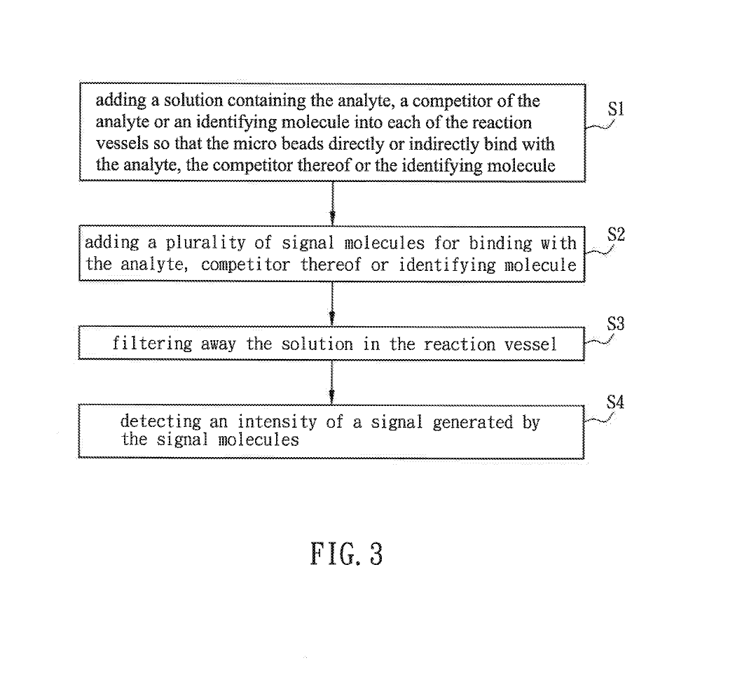

The analysis method of the invention can be applied to the above-mentioned assay kit, and the reaction vessel and components of the assay kit has been described hereinabove. The steps and operations of the analysis method of the invention are shown in FIGS. 3 and 4A to 4E. Herein, FIGS. 4A to 4E show the assay kit of FIG. 1B applied to a competitive ELISA.

In the step S1 of one embodiment, a competitor that can compete with the analyte is directly bind with the micro bead. Herein, the micro beads are added into the reaction vessel in advance, and then the solution containing the competitors is added and reacts with the micro beads for a whole, so that the competitors can be fully bound with the micro beads.

In another embodiment, the competitor indirectly bound with the micro bead. Herein, the analysis method of the invention further includes a step of disposing at least a first ligand on the surface of the micro head. The first ligand directly or indirectly connects the analyte or its competitor to the micro bead. In the step S1, the micro bead bound with the first ligand is directly disposed in the reaction vessel, and then the solution containing the analyte or its competitor is added into the reaction vessel and bound with the micro bead. If the analyte or its competitor is indirectly bound with the micro bead, the analysis method may further include a second ligand disposed on the analyte or its competitor. The first ligand can bind with the second ligand by any of the above mentioned ways. With reference to FIGS. 3 and 4A, in this embodiment, the micro bead 12 is bound with the first ligand L1 and then disposed in the reaction vessel 11, and the competitor A1 is bound with the second ligand L2, FIG. 4A shows a single reaction vessel 11 for example. In practice, multiple reaction vessels 11 are configured for an analysis. Except for the above arrangement, the step S1 of the present invention may be to bind the first ligand L1 with the competitor A1 or the second ligand L2, and then connect to the micro bead 12 through the first ligand. Of course, in other embodiments, the micro bead 2 may directly or indirectly connect with an analyte A.

In this embodiment, the micro bead 12 is made of resin, the first ligand L1 is streptavidin, and the second ligand L2 is a biotin, which can be engaged with the first ligand L1 (streptavidin), Herein, the micro bead 12 is immersed in a solution containing streptavidin, so that the streptavidin is bonded on the surface of the micro bead 12. The competitor A1 can be connected with the first ligand L1 through the second ligand L2, and can compete the specific identifying molecule 14 (see FIG. 4B) against the analyte A. To be noted, the analyte A and the competitor A1 can compete with the identifying molecule 14 by different ways depending on the analysis purpose. For example, the analyte A bound with the second ligand L2 is connected with the first ligand L1 of the micro bead 12, and then the competitor A1 is added. Otherwise, the competitor A1 bound with the second ligand L2 is added so the second ligand L2 connects with the first ligand L1 of the micro bead 12, and then the competitor A1 is added. Alternatively, the analyte A and the competitor A1 are mixed and added, and then the competitor A1 is bound with the second ligand L2 for connecting with the first ligand L1 of the micro bead 12. The competitor A1 of the invention plays a competitive role according to the requirement of the analysis, and this invention is not limited. As shown in FIG. 4A, after the second ligand L2 bound on the competitor A1 is connected with the first ligand L1, the analyte A is added into the reaction vessel 11 for competing with the later identifying molecules 14.

In this embodiment, the second ligand L2 is a biotin. For marking the analyte A or the competitor A1, Sulfo-NHS-LC-LC-Biotin (20 times of antibiotics) is mixed in the solution containing the competitor A1. After reacting for 2 hours at room temperature, 68 mM bovine serum albumin (BSA) is added, so the residual Sulfo-NHS-LC-LC-Biotin is reacted with the amino group of BSA. Then, tris-HCI is added to bond with the residual NHS reaction functional groups, and the biotin binding with the competitor A1 and the biotin binding with BSA are separated by amicon ultrafiltration (Millipore) based on their different sizes. The filtrate containing the competitor A1 marked by biotin is added with 1.5 times of micro beads 12 bound with streptavidin. After reacting for 1 hour at room temperature, the biotin and the streptavidin are fully bonded. At the same time, the BSA solution is added to block the specific absorption of the surface of the micro bead 12. Finally, the mixture is centrifuged and washed to remove the other substances (e.g. ionized competitor A1) that are not bound onto the micro bead 12. After the centrifugation and washing for several times, the prepared micro beads 12 bound with the competitor A1 is stored at 4° C. for the following procedures.

In the step S2, as shown in FIG. 4C, a plurality of signal molecules S are added. In this embodiment, the signal molecule S is encapsulated by a liposome to form a signal complex 15. Herein, the signal complex 15 can be specific bound with or marked to the analyte A or its competitor A1 through an identifying molecule 14. In order to improve the specific binding of the signal complex 15 and the identifying molecule 14, the signal complex 15 further includes a third ligand L3, which has better specific binding with the identifying molecule 14. Thus, the signal complex 15 carrying the signal molecule S can be more effectively bound with the analyte A or its competitor A1 containing the identifying molecule 14 indirectly. By the connection of the identifying molecule 14 and the third ligand L3, the signal molecules S can mark the analyte A or its competitor A1. Of course, in other embodiments, the signal complex 15 may be directly bound with the analyte A and/or its competitor A1 without the identifying molecule 14 or the third ligand L3.

In this embodiment, the signal molecule S is a fluorescent substance SRB (sulforhodamine B, Sigma) encapsulated in the liposome, which can be prepared by the following method. The liposome is composed of 4.8% DPPG (dipalmitoylphosphatidylglycerol, Avanti Polar Lipids), 45.3% DPPC (dipalimitoylphosphatidylcholine, Avanti Polar Lipids), 45.9% cholesterol (Sigma), and 4% ATA-DPPE. The Liposome encapsulates 150 mM SRB (sulforhodamine B, Sigma). DPPE-ATA is prepared by mixing and reacting N-Succinimidyl S-Acetylthioac tate (SATA, Thermo) and DPPE (1,2-dihexadecanoyl-sn-glycero-3-phosphoethanola-mine, Avanti Polar Lipids) for 30 minutes. Afterwards, cholesterol, DPPC and DPPG are dissolved in an organic solvent (3:1 chloroform/methanol), and then the prepared DPPE modified by SATA is added. After removing the organic solvent by nitrogen, a colloid transparent lipid film is obtained followed by adding preheated (60° C.) 150 mM SRB solution. The encapsulation reaction of fluorescent substance SRB is performed at 60° C. water bath for 45 minutes. Then, the residual solid lipid is shaken and uniformly mixed in the solution. The encapsulation reaction of fluorescent substance SRB is continuously performed at 60° C. water bath for 30 minutes to 1 hour.

Finally, the prepared fluorescent liposome solution is filtered through a polycarbonate syringe filers (200 nm mesh, Avanti Polar Lipids) for 30 times at 60° C. Accordingly, the diameter of the fluorescent liposome is adjusted to approximate 200 nm. The residual fluorescent substance SRB and the liposome encapsulating the fluorescent substance SRB are separated by size exclusion chromatography (SEC). The liposome encapsulating the fluorescent substance SRB can be separated by SEC (CL-4B resin) with the elution of TBS-sucrose (25 in M Tris, 140 mM NaCl, and 65 g/L sucrose, pH 7.5).

In this embodiment, the third ligand L3 is a protein (1; and the identifying molecule 14 is an immunoglobulin G (IgG), which is an antibody capable of specific binding with the analyte A and its competitor A1. Since the protein G can be specific bound with the immunoglobulin G, the signal complex 15 can specific bind with the identifying molecule 14 through the third ligand L3.

The protein G can be connected to the surface of the signal complex 15 by the modified conjugation method disclosed by Chen et al. (2005), which substitutes the original Sulfo-SMCC (Sulfosuccinimidyl 4-(N-maleimidomethyl)cyclohexane-1-carboxylate) crosslinker with SM(PEG)₂₄ (succinimidyl-[(N-maleimidopropi-onamido)-tetracosa ethyleneglycol]ester) crosslinker (Thermo) containing 24 PEG. Accordingly, protein G-liposome-SRB can break through the spatial disorder and have maximum freedom for binding with the identifying molecule 14 (antibody) in the following analysis procedures.

The procedures of the conjugation of the protein G and the liposome of the signal complex 15 will be described hereinafter. Firstly, the derivitization of neutravidin with SM(PEG)₂₄ and the deacetylation of the surface of the liposome-fluorescent substance SRB are simultaneously performed. Herein, the derivitization of the protein G is performed at room temperature and pH 7-9 for 1 hour, and the reaction is terminated by 0.2M Tris HCl. The protein G binding with SM(PEG)₂₄ and the non-reacted SM(PEG)₂₄ are separated by a centrifugal column (Zeba Desalting column) so as to obtain the Maleimide-derivetized protein G. The deacetylated liposome by 0.5 M hydroxylamine hydrochloride at room temperature for 1 hour to expose the surface thiol group is added to react with the Maleimide-derivetized protein G at pH 6.5-7.5 and room temperature for 1.5 hours. NEM (Wethylmaleimide) is added to block the non-reacted thiol group of the liposome. Then, the residual protein G and the liposome-fluorescent substance SRB binding with the protein G are separated by size exclusion chromatography (TBS-sucrose, 25 mM Tris, 140 mM NaCl, and 65 g/L sucrose, pH 7.5).

After the steps S1 and/or S2, as shown in FIGS. 3 and 4D, a filtering step (step S3) is carried out. After the steps S1 or S2, a sealer 112 is provided to seal the pores P of the filter membrane 111, so that the added substances can fully react and bind with each other. Then, the sealer 112 is removed to filter away the unbound substances, such as the analyte A, competitor A1, first ligand L1, second ligand L2, identifying molecule 14 or signal complex 15, through the pores P of the filter membrane 111. Besides, the filtering step may further include several washing procedures to significantly remove the non-targeted substances.

Finally, the step S4 is to detect a signal intensity of the signal molecules S of the signal complex 15 that binds with the competitor A1 of the micro bead 12. According to different signal molecules S, the analysis method of the invention may add additional substrates or enzymes to activate the signal molecules S so as to release the signals. In this embodiment, as shown in FIG. 4E, a surface active agent is added to dissolve the liposome of the signal complex 15 so as to release the SRB signal molecule S. Then, the concentration of the analyte A can be quantified according to the measurement results of the wavelength or signal characteristics of the signal molecule S.

To be noted, the more the analytes A are contained in the solution, the I the amounts of the identifying molecules 14 and the signal molecules S are bound with the competitors A1. On the contrary, the less the analytes A are contained in the solution, the more the amounts of the identifying molecules 14 and the signal molecules S are bound with the competitors A1. Besides, the analytes A that are unbound with the micro beads 12 will be filtered away in the step S3, so that the detected intensity of the signal molecules S remained in the reaction vessels 11 is in inverse proportion with the concentration of the added analytes A. Accordingly, the concentration of the analytes A can be estimated indirectly.

In a sandwich ELISA, the solution added into the reaction vessels further contains at least one identifying molecule for specific binding with the analyte or its competitor and for directly or indirectly binding with the micro beads. Similarly, with reference to FIGS. 3, 1C and 1D, the identifying molecule 13 can specific bind with the analyte A. In this embodiment, the identifying molecule 13 is an antibody that can bind with the analyte A. As shown in FIG. 1C, the identifying molecule 13 is directly bound with the surface of the micro bead 12. Alternatively, in order to firmly bind the identifying molecule 13 on the micro bead 12, the micro bead 12 is directly bound with a first ligand L1 and the identifying molecule 13 is bound with a second ligand L2 before the step S1 (see FIG. 1D). The connection methods of the first ligand L1 and the second ligand L2 have be described hereinabove, so the detailed descriptions thereof will be omitted hereinafter. However, different first ligands L1 and different second ligands L2 may be bound by different ways or means. After the identifying molecule 13 is directly or indirectly bond with the micro bead 12, the analyte A or its competitor A1 is added for binding with the identifying molecule 13, thereby directly or indirectly binding the identifying molecule 13 on the micro bead 12 (step S1). The following steps S2, S3 and S4 for the sandwich ELISA are the same as the previously mentioned procedures, so the detailed descriptions thereof will be omitted.

The analysis method of the invention capable of rapidly and precisely building the standard curve for detection and analyzing and executing multiple analyzing will be described hereinafter.

### Example

### Application of Analysis Method of the Invention in Competitive ELISA for Analyzing Antibiotics

In this example, the elements and connections of the adopted assay kit are shown in FIG. 4A, wherein the micro bead 12 is a particle made of resin. The analyte A and its competitor A1 are both gentamycin sulfate, and a plurality of first ligands L1 disposed on the micro bead 12 are streptavidin. The gentamycin sulfate can mark biotin and then be bound with the streptavidin of the micro bead 12. In this embodiment, the reaction vessel 11 is a filter plate.

Firstly, bovine serum albumin (BSA) is added for blocking all possible non-specific absorption in the filter plate. Then, the gentamycin sulfate micro beads and six gentamycin sulfate samples with different concentrations of 0 (negative control sample), 0.05, 0.1, 1, 10, and 100 μg/mL are added into the filter plate with bottom sealer. The added materials are well mixed by a micromixer (TAITEC) for several seconds, and then the needed anti-gentamycin antibody solution is added at room temperature for 1 hour. After the reaction, the sealer 112 (e.g. a foil) is reomoved, and the TBS-sucrose is added into the filter plate for washing (3 times) out the residual anti-gentamycin antibody-gentamycin sulfate, anti-gentamycin antibody and gentamycin sulfate (except for the gentamycin sulfate micro beads bound with or without the anti-gentamycin antibody). After the washing procedures, the filter plate is centrifuged to remove the redundant liquid. The bottom of the 96-well filter plate is sealed by foil again, and the solution containing the signal complex (protein G-liposome-SRB signal molecule) is added. After 30 minutes, the sealer 112 is removed and the filter plate is washed twice by TBS-sucrose solution again to remove the redundant protein G-liposome-SRB. Finally, the surface active agent n-OG (noctyl-beta-d-glucopyranoside) is added to the 96-well filter plate to break the protein G-liposome-SRB, and then the filter plate is centrifuged to inject the SRB released from the broken protein G-liposome-SRB into a normal 96-well black plate. The intensity of the fluorescent signal is measured by synergy 2 (Synergy 2 Multi-Mode microplate reader, BioTek) under 540 nm excitation spectrum and 590 nm absorption spectrum followed by proper statistics analysis.

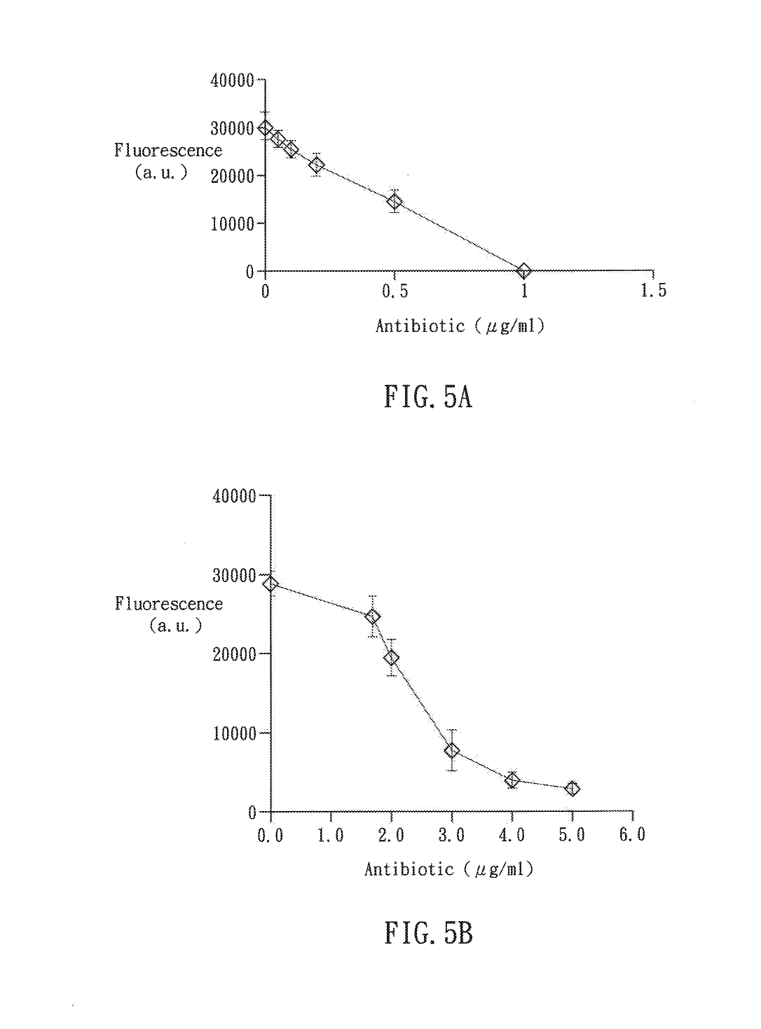

According to the analysis method of the invention, the gentamycin sulfate of six different concentrations (5 repeats) are sampled on 30 wells of a filter plate for performing a high throughput filter plate bead based competitive assay. Herein, the six gentamycin sulfate samples with different concentrations of 0 (negative control sample), 0.05, 0.1, 0.2, 0.5, and 1 μg/mL (in TBS, Tris buffered saline) are provided, and the result products are collected in a normal 96-well black plate. The measured fluorescent signals of the 96-well black plate are as shown in FIG. 5A, wherein the measured result by the assay kit and analysis method of the invention has a stable linear relation (R²=0.995). In another example, the samples are prepared by dissolving the gentamycin sulfate of different concentrations in skim milk. Following the above steps, the measured result also has a good linear relation (R²=0.9314). As a result, the assay kit and analysis method of the invention have excellent specificity and may not be affected by other substances in the sample solutions. Moreover, after obtaining a stable standard curve and providing specific analyzing conditions, the present invention can further detect the analyte of unknown concentration to obtain accurate detection results.

In order to detect saturated dose response curve, different concentrations of 0 (negative control sample), 0.05, 0.1, 1, 10, and 100 μg/mL are selected. As shown in FIG. 5B, the limit of detection (LOD) of a certain concentration is defined as the average of the negative control samples subtracted by three times of the standard deviation (SD) of the negative control samples. The calculated LOD in TBS buffer and skim milk are 52.65 ng/mL and 14.16 ng/mL, respectively, which is sufficient for detecting the Codex maximum residue level (MRL) of gentamycin sulfate (100 ng/mL). Besides, the assay kit and analysis method of the invention can rapidly detect 30 standard samples within 2 hours, so the invention can achieve rapid and precise analyzing.

To sum up, in the assay kit of the invention, the diameter of each micro bead is greater than that of each pore, so that the beads directly or indirectly binding with the analyte, the competitor thereof or the identifying molecules can be rapidly separated by filtering for the following quantitative analysis. Thus, the complex procedures for providing magnetic force to separate the magnetic beads in the conventional art can be omitted. The assay kit of the invention also has the advantages of easily analyzing, lower equipment cost, and applicable to various analysis methods. Besides, the reaction vessels can be designed in different sizes according to the desired analysis, so they can be applied to high throughput analyze. In addition, the present invention also discloses an analysis method applied with the above-mentioned assay kit, which adapts to indirect ELISA, competitive ELISA, and sandwich ELISA. Compared with the conventional analysis method, the analysis method of the invention can more rapidly and sensitively analyze and quantify the analyte.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An assay kit for reacting with an analyte, comprising:
a plurality of reaction vessels, each of which contains a filter membrane with a plurality of pores; and
a plurality of micro beads, wherein the diameter of each of the micro beads is greater than that of each of the pores, and each of the micro beads is directly or indirectly bound to the analyte, a competitor of the analyte or an identifying molecule.

2. The assay kit of claim 1, wherein the diameter of each of the pores is between 1 nm and 1 cm.

3. The assay kit of claim 1, wherein the beads are made of glass, latex, rubber, magnet, resin, metal, ceramic, polysaccharide, plastic, or silicon.

4. The assay kit of claim 1, wherein the analyte, the competitor thereof or the identifying molecule is protein, peptide, nucleic acid, saccharide, compound, cell, or microorganism.

5. The assay kit of claim 1, wherein the identifying molecule is configured for binding with the analyte or the competitor thereof.

6. The assay kit of claim 1, further comprising:
a first ligand for directly or indirectly binding the analyte, the competitor thereof, or the identifying molecule on the beads.

7. The assay kit of claim 6, further comprising:
a second ligand for connecting with the analyte, the competitor thereof, or the identifying molecule, wherein the second ligand is bound with the first ligand by antigen-antibody binding, protein-cofactor binding, nucleic acid-nucleic acid binding, nucleic acid-saccharide binding, nucleic acid-compound binding, protein-protein inhibitor binding, protein-saccharide binding, protein-lipid binding, protein-compound binding, enzyme-enzyme receiver binding, or protein-nucleic acid binding.

8. The assay kit of claim 1, further comprising:
a plurality of signal molecules for binding with the analyte, the competitor thereof or the identifying molecule.

9. The assay kit of claim 8, wherein the signal molecule comprises enzyme, enzyme substrate, chromogenic agent, radioactive substance, nano-lipid particle, or metallic compound.

10. The assay kit of claim 1, wherein the reaction vessel comprises at least a filter plate or at least a column.

11. The assay kit of claim 1, further comprising:
a sealer disposed at a downstream of the filter membrane.

12. An analysis method applied to an assay kit for reacting with an analyte, wherein the assay kit comprises a plurality of reaction vessels and a plurality of micro beads, and each of the reaction vessels contains a filter membrane with a plurality of pores, the analysis method comprising the steps of:
adding a solution containing the analyte, a competitor of the analyte or an identifying molecule into each of the reaction vessels so that the micro beads directly or indirectly bind with the analyte, the competitor thereof or the identifying molecule;
adding a plurality of signal molecules for binding with the analyte, the competitor thereof or the identifying molecule;
filtering away the solution in the reaction vessel; and
detecting an intensity of a signal generated by the signal molecules.

13. The analysis method of claim 12, wherein the diameter of each of the micro beads is greater than that of each of the pores.

14. The analysis method of claim 12, wherein at least one of the identifying molecule binds with the analyte or the competitor thereof and directly or indirectly binding with the micro beads.

15. The analysis method of claim 14, further comprising a step of:
disposing at least a first ligand on the surfaces of the micro beads, wherein the first ligand directly or indirectly binds with the analyte, the competitor thereof, or the identifying molecule.

16. The analysis method of claim 15, further comprising a step of:
disposing a second ligand on the analyte, the competitor thereof, or the identifying molecule, wherein the second ligand is bound with the first ligand.

17. The analysis method of claim 16, wherein the second ligand is bound with the first ligand by antigen-antibody binding, protein-cofactor binding, nucleic acid-nucleic acid binding, nucleic acid-saccharide binding, nucleic acid-compound binding, protein-protein inhibitor binding, protein-saccharide binding, protein-lipid binding, protein-compound binding, enzyme-enzyme receiver binding, or protein-nucleic acid binding.

18. The analysis method of claim 12, further comprising a step of:
disposing a sealer at a downstream of the filter membrane.

19. The analysis method of claim 18, further comprising a step of:
removing the sealer to allow the solution to flow through the pores.
